(19) United States
(12) Patent Application Publication
(10) Pub. No.: US 2006/0051749 A1
(43) Pub. Date: Mar. 9, 2006

(54) Polymorphism and haplotype scoring by differential amplification of polymorphisms

(75) Inventors: Yan Wang, San Francisco, CA (US); Lei Xi, Foster City, CA (US); Michael Finney, San Francisco, CA (US); Fan Chen, Fullerton, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: MJ BIOWORKS INCORPORATED, South San Francisco (CA)

(21) Appl. No.: 10/496,702

(22) PCT Filed: Nov. 27, 2002
(86) PCT No.: PCT/US02/38278

§ 371 Date: Oct. 29, 2004

Related U.S. Application Data
(60) Provisional application No. 60/334,046, filed on Nov. 28, 2001.

(51) Int. Cl. C12Q 1/68; C12P 19/34

(52) U.S. Cl. 435/6; 435/91.2

(57) ABSTRACT

The current invention provides a method of performing DNA polymorphism assays. The assay combines allele-specific PCR with technology used for quantitative PCR. The method can be used to score the presence of absence of particular polymorphisms in a DNA sample. In a further aspect of the invention, the method is used to score the presence or absence of particular haplotypes in a DNA sample.

### CROSS-REFERENCES TO RELATED APPLICATIONS

This application claims the benefit of U.S. provisional application No. 60/334,046, filed Nov. 28, 2001, which is incorporated by reference herein.

### FIELD OF THE INVENTION

The current invention provides a method of performing DNA polymorphism assays, in which a specific assay for a particular polymorphism can be set up for the cost of synthesizing three or four primer oligonucleotides, run with little more difficulty and for little more marginal cost than two small-scale PCR reactions, using only common laboratory equipment The assay combines allele-specific PCR with some of the technology used for quantitative PCR, along with the surprising observation that simple rules are sufficient to design reliable assays with little or no experimentation. The method of the invention may be referred to as DAP, for Differential Amplification of Polymorphisms. In one aspect of the invention, DAP is used to score the presence or absence of particular polymorphisms in a DNA sample. In a further aspect of the invention, DAP is used to score the presence or absence of particular haplotypes in a DNA sample.

### BACKGROUND OF THE INVENTION

Single Nucleotide Polymorphisms and Haplotypes

The smallest possible difference between two DNA sequences is a change of a single base, a Single Nucleotide Polymorphism or SNP. Such differences are common in the human population, occurring roughly one every 1000 bases between any two unrelated individuals. Some SNPs have medically important consequences, while others are silent but may be useful as markers to study genetic transmission of traits.

A number of methods have been developed to score SNPs, including allele-specific hybridization, electrophoretic DNA sequencing, single-nucleotide extension using labeled chain terminators, the “Invader” assay (Third Wave Technologies, Madison Wis.), mass spectrometry, the 5′ nuclease assay (Taqman; see below), etc. All of these methods entail assays that are either difficult or expensive to develop, or difficult or expensive to perform.

It will be appreciated that while SNPs are common, it is at times advantageous to score other polymorphisms such as insertions, deletions, rearrangements or sequence alterations involving more than one base. SNP scoring has been emphasized in the literature because it is the most difficult case, but essentially any method capable of scoring SNPs is also capable of scoring additional types of polymorphisms.

Although scoring of SNPs is important in medicine and research, scoring of haplotypes can, in some situations, provide information that is not available from SNP scoring. Minimally, a haplotype consists of at least two polymorphisms linked together on the same chromosome, where each of the polymorphisms can at least conceptually occur independently of the other.

The importance of haplotypes is apparent, e.g., in the following situation of a single gene that is known to have two SNPs at two different sites, wherein each of the SNPs has an allele that is compatible with gene activity (a sense allele) and an allele that destroys gene activity (a nonsense allele). If a diploid individual is scored for both SNPs, and it is found that the individual has both a sense and nonsense allele at both sites (the only information that can be obtained by scoring SNPs) the information is insufficient to determine whether the individual has a functional copy of this gene. That is, the individual may have both nonsense alleles in one copy of the gene, and both sense alleles in the other copy. The second copy is functional, and therefore the individual has gene function. Alternatively, the individual may have one sense and one nonsense allele in each copy of the gene. In this case, the individual has no functional copies of the gene, and may display a mutant phenotype.

Recently Stephens et al. (Science 293(5529):489-93, Jul. 20, 2001) studied the pattern of inheritance of SNPs in human genes, and found that, due to linkage disequilibrium, SNPs are naturally organized into haplotypes, and that these haplotypes carry more useful genetic information (heterozygosity) than the SNPs themselves. Haplotypes may serve as markers for particular phenotypes, even if the causal mutation is not identified or understood. Scoring of such haplotypes can be medically important, and scoring of the individual polymorphisms that make up the haplotypes would not suffice.

It is thus important to distinguish haplotypes, and to date, most methods of scoring SNPs are inadequate for the job. Only techniques that preserve the information of whether two polymorphisms are linked within the same molecule will work. One such technique is molecular cloning of DNA segments from the individual followed by analysis of individual clones, but this method is very expensive and labor intensive.

Allele-Specific PCR

Allele-specific PCR (ASP; also called Amplification-Refractory Mutation System or ARMS) has been used since at least 1989 (Newton et al., Nucl. Acids. Res. 17:2503-16, 1989). In its simplest form, ASP consists of two separate PCRs performed with a common reverse primer and two respective forward primers differing at the terminal base, so that each of the forward primers is a perfect match to one of two SNP alleles, and has a single terminal mismatch with the other SNP allele. The two PCRs are completed, and the amount of product produced by each is compared.

ASP depends on the reduced rate of extension by polymerases from a primer with a 3′ mismatch with the template (mis-extension). Most of this discrimination occurs at the beginning of the PCR, because after the first five cycles, more product will be generated by amplification of previously mis-extended products than will be generated from mis-extension of templates with the original sequence. After this point, the reaction proceeds essentially as a normal PCR, albeit with greatly reduced starting template amount.

Scoring of ASP has depended on the expectation that a larger amount of product will be generated for the perfectly-matched case than for the mismatch. Thus, for a given sample, if the yield of one reaction is significantly greater than the other, the reaction with greater yield is assumed to correspond to the perfect match. If the yields are approximately equal, the sample is called as a heterozygote.

ASP has failed to become a dominant method of scoring SNPs, e.g., a recent review of practical SNP scoring technologies does not mention ASP (M. Shi, Clinical Chemistry 47(2): 164-172, 2001) because ASP as it is practiced in the prior art is not reliable. ASP is not reliable because the fidelity of known thermostable enzymes is limited, so that a product will always be produced in a mismatch condition, and any of several conditions can result in the product from the mismatch condition being produced in amounts roughly equal to those produced from the perfect match condition.

For instance, it is known that some of the 12 possible types of mismatch result in less discrimination than others by a factor of up to 10⁴ (Huang et al., Nucl. Acids Res. 20(17) 4567-4573, 1992). Furthermore, the efficiency of discrimination depends greatly on the exact sequence context of the mismatch and a number of other conditions, not all of which are well-defined (Ayyadevara et al., Anal. Biochem. 284:11-18, 2000). Furthermore, as the amount of product increases in a PCR, the efficiency of amplification begins to decrease, until the reaction reaches a “saturation” point where very little more product is produced with additional cycles. Major factors in decrease in amplification efficiency are enzyme limitation and inhibition by reannealing of products. This decrease in amplification efficiency with increasing product amount will strongly decrease the difference in amount between perfect match and mismatch conditions as the reaction proceeds. In any ASP, if the reaction is continued for a sufficient number of cycles, both match and mismatch conditions will produce a saturating amount of product, and there will be no difference between them. Prior-art methods of quantifying products, such as gel electrophoresis, have depended on the presence fairly large amounts of product, and therefore have depended on continuing the reactions past the point where the differences between the mismatch and perfect match conditions begin to decrease.

Overcoming Difficulties with ASP

The above problems would be surmountable if the PCR were stopped at exactly the right point, where sufficient product has accumulated in the perfect match condition for scoring, but not so much product has accumulated in the mismatch condition to confuse the result. However, at least three factors make this precision extremely difficult to achieve.

First, the amount of input sample DNA must be well controlled. If too much sample DNA is added, the reactions will reach saturation in a smaller number of cycles, and the mismatch condition reaction will “catch up” to the perfect match condition. If too little DNA is added, not enough of the perfect match product will be produced. Adding an exactly measured amount of DNA is very difficult to achieve for samples such as clinical samples.

Second, the efficiency of PCR must be very similar for each sample. Even if the amount of DNA is well-controlled, if samples contain varying amounts of inhibitors, then some reactions may saturate while others have insufficient yield. The presence of varying amounts of inhibitors such as heme is a significant issue for clinical DNA samples purified from tissues such as blood.

Third, the efficiency of PCR must be the same for each different SNP assay to be run in parallel. Because of differences in template and primer melting temperatures, and other factors such as secondary structure in amplicons, some PCRs have lower amplification efficiency than others with a given set of conditions. Differing efficiencies can cause some assays to saturate in fewer cycles than others, making ASP difficult to score. Thus, even with well-controlled sample DNA concentration and purity, a requirement to run different assays in parallel makes ASP unreliable.

Previous attempts to increase the reliability of ASP have generally involved methods of increasing the discrimination against mis-extension, so that there will be a greater margin of error for the number of cycles to be run before scoring. In particular, it has become common practice to introduce a second mismatch between primer and template, such that the “match” condition has a single mismatch and the “mismatch” condition has two nearby mismatches. While this decreases the efficiency of the “match” condition, it can increase the relative efficiencies, thereby giving additional margin of error. The difficulty with this approach is that there is no clear rule for designing primers, and experimentation and optimization may be required to achieve the desired results.

Quantitative PCR

“Quantitative,” “real-time,” or “kinetic” PCR is a method of determining the concentration of an initial template DNA by determining how many PCR cycles are required to generate a threshold value of amplification-dependent signal (the “threshold cycle” or Cₜ), relative to standard template concentrations. Automation of quantitative PCR (QPCR) is possible if the amplification-dependent signal can be read out each cycle non-destructively.

QPCR automated by fluorescent reading is described in U.S. Pat. Nos. 5,994,056 and 6,171,785, and use of the dye SYBR Green I for this purpose is described in Morrison et al. (Biotechiques 24, 954-962, 1998). SYBR Green I (Molecular Probes, Inc., Eugene, Oreg.) is described in U.S. Pat. Nos. 5,436,134 and 5,658,751.

In addition, several other fluorescent readout methods for QPCR exist, such as “Taqman,” described in U.S. Pat. Nos. 5,210,015 and 5,487,972.

Self-quenched primers can also be used for QPCR. Such oligonucleotides are designed and synthesized such that when the oligonucleotide is not paired with its complement, the fluorophore has lower fluorescence relative to the fluorophore of the same oligonucleotide when it is paired with its complement. For instance, an oligonucleotide may be synthesized so that a fluorophore is attached at or near one end, and a quencher is attached at a distance of at least several nucleotides from the fluorophore (for instance, at or near the opposite end of the oligonucleotide). The quencher may be a contact type such as 4-(4′-dimethylaminophenylazo) benzoic acid (DABCYL) or a FRET type such as TAMRA or Black Hole Quencher (Biosearch Technologies, Novato Calif.). The quencher may also be a feature of the DNA itself, such as guanosine residues (Nazarenko et al., Nucleic Acids Res. 30:2089-2095, 2002). Inclusion of the primer into a double-stranded nucleic acid physically separates the fluor from the quencher, resulting in a significant increase in fluorescence. When self-quenched oligonucleotides are used as primers in a PCR, an increasing amount of the input primer is incorporated into double-stranded product with each cycle, resulting in an increasing fluorescent signal that can be used to determine the amount of input template. The primer oligonucleotide need not be incorporated into a complete double-stranded molecule, or need not be paired with a complete complement, as long as a sufficiently large portion of the oligonucleotide is paired with a complement so as to separate the fluor and quencher sufficiently to produce a detectable signal. Examples of self-quenched primers include Nazarenko et al., Nucleic Acids Res. 25:2516-2521, 1997; U.S. Pat. Nos. 5,866,336, 6,090,552, and 6,117,635; Thelwell et al., Nucleic Acids Res. 28:3752-3761, 2000; and U.S. Pat. Nos. 6,277,607 and 6,365,729.

Using OPCR for Scoring SNPs

Several groups have attempted to overcome the difficulties with ASP by combining it with QPCR or fluorescent readout. The idea is that by recording the cycle number at which the amplification-dependent signal reaches the C, for each of the match and mismatch conditions, it should be possible to distinguish the two. Differing amounts of input template DNA, differing amplification efficiencies, etc., will affect the match and mismatch conditions in similar ways, so that while the actual Cₜ may vary, the difference in Cₜ (ΔCₜ) between match and mismatch conditions will be relatively constant.

For instance, Whitcomb et al. (Clin. Chem. 44(5): 918-923, 1998) used ASP in combination with Taqman detection. In each case, there were at least two differences between the two primers near their 3′ ends. In addition, Taqman probes are expensive if made specifically for the reaction, or, as in this case, require the synthesis of very complicated primers containing Taqman probe binding sites.

In another variation, Hiratsuka et al. (Mol. Gen. Metab. 68:357-363, 1999) perform ASP using SYBR Green I as a readout. Match and mismatch primers each have an additional mismatch separate from the SNP site, so that the re are two differences between the mismatch primer and the template.

Germer et al. (Genome Res. 10(2): 258-266, 2000) use a combination of ASP with primers differing only at the 3′ base (corresponding to the SNP site) and SYBR Green I detection, but they do not use it to score SNPs in individual samples. Instead, the protocol is used to estimate allele frequency differences among populations.

Donohoe et al. (Clin. Chem. 46(10): 1540-1547, 2000) use a version of ASP called mutation-separated PCR in which additional mismatches to the template sequence are introduced into each primer, in combination with 5′ extension of one of the primers, so that both SNP alleles can be amplified in the same tube, and the products distinguished by SYBR Green I melting curve analysis post-amplification. Because both alleles are amplified in the same tube, they are not able to measure the amplifications individually using QPCR.

Higuchi in U.S. Pat. No. 5,994,056 describes a method of distinguishing between alleles differing by a single nucleotide using ASP, with readout by measuring fluorescence of the dye ethidium bromide incorporated into the reaction buffer. The assay is an end-point assay only, with fluorescence being measured only after completion of the PCR cycles. Each tube is scored as + or − depending on whether a detectable signal is present or not. There is no suggestion that the − tubes would eventually produce a product or that allele scoring could be based on any difference in the number of cycles needed to produce a product.

Thus, the prior art methods of scoring SNPs and other polymorphisms are deficient. The current invention provides a methods of analyzing polymorphisms that provides the ability to determine haplotypes, i.e., whether the polymorphisms in question, e.g., SNPs, are on the same, or different, chromosomes.

In some embodiments, self-quenched primers may be used. Self-quenching primers have been used in allele-specific PCR (Nazarenko el al., Nucleic Acids Res. 30:2089-2095, 2002), but not with more than one allele in a single reaction, and only as an end-point assay, as opposed to QPCR.

Thus, there is a need to develop polymorphism detection systems that can be multiplexed and are efficient. The current invention addresses that need. In some embodiments, it can be advantageous to use a polymerase with enhanced processivity. Co-pending U.S. application Ser. No. 09/870,353 discloses modified polymerases the efficiency of the enzyme increased by joining a sequence-non-specific double-stranded nucleic acid binding domain to the enzyme, or its polymerase domain.

### BRIEF SUMMARY OF THE INVENTION

The invention provides a method of scoring polymorphisms. Further, the invention provides a method of scoring haplotypes.

In one aspect, the invention provides a method of distinguishing the presence or absence in an input sample containing DNA of at least two alternative DNA sequence elements, where those elements are at least seven based in length, typically ten bases in length, and differ in sequence in at least one position. The method requires the performance of at least two polymerase chain reactions, where: a first reaction comprises said input sample containing DNA, a first forward primer with a 3′ end having at least seven bases, typically ten bases, that are exactly complementary to a first allele of a DNA sequence element, and a reverse primer capable of participating with the first forward primer in a polymerase chain reaction; and a second reaction comprises said input sample containing DNA, a second forward primer with a 3′ end that has at least seven bases, typically at least ten bases, that are exactly complementary to a second allele of a DNA sequence element, and a reverse primer capable of participating with the second forward primer in a polymerase chain reaction; and in conjunction with the performance of said at least two polymerase chain reactions, monitoring of the amount of amplicon produced in each reaction by measuring fluorescent emission from a quantitation reagent sensitive to the amount of double-stranded polynucleotide present in the reaction, where such monitoring occurs automatically no less often than every fifth polymerase chain reaction temperature cycle, and comparing the amount of fluorescence in each reaction to determine whether the corresponding allele is present in the input sample containing DNA.

In a second aspect, the invention provides a method of distinguishing the presence or absence in an input sample containing DNA of at least two alternative DNA sequence elements, where those elements are at least seven bases in length, typically ten bases in length, and differ in sequence in at least one position, using only a single reaction in a single sample vessel. In this method, at least two self-quenched primers with different and distinguishable attached fluorophores are used as the allele-specific primers. The reaction comprises said input sample containing DNA, a first forward self-quenched primer whose 3′ end has at least seven and typically at least ten bases that are complementary to, and a 3′ terminal base exactly complementary to, a first allele of a DNA sequence element; a second forward self-quenched primer whose 3′ end has at least seven bases and typically at least ten bases that are complementary to, and a 3′ terminal base exactly complementary to, a second allele of a DNA sequence element; and a reverse primer capable of participating with the first and second forward primers in a polymerase chain reaction; and in conjunction with the performance of said polymerase chain reaction, monitoring of the amount of amplicon produced using each self-quenched primer by measuring fluorescent emission from each self-quenched primer, where such monitoring occurs automatically no less often than every fifth polymerase chain reaction temperature cycle, and comparing the amount of fluorescence corresponding to each self-quenched primer to determine whether the corresponding allele is present in the input sample containing DNA.

In another aspect, the invention provides a method of distinguishing the presence or absence in an input sample containing DNA of at least two allelic DNA sequence haplotype elements, where those haplotype elements are at least twenty bases in length and differ in sequence in at least two positions. The method requires the performance of at least two polymerase chain reactions, where: a first reaction comprises said input sample containing DNA, a first forward primer whose 3′ end has at least seven bases and typically at least ten bases that are complementary to, and a 3′ terminal base exactly complementary to, a first region of a first allelic DNA sequence haplotype element, and a first reverse primer whose 3′end has at least seven bases and typically at least ten bases that are complementary to, and a 3′ terminal base exactly complementary to, a second region of said first allelic DNA sequence haplotype element, where said first forward primer and said first reverse primer are capable of participating together in a polymerase chain reaction; and a second reaction comprises said input sample containing DNA, a second forward primer whose 3′end has at least seven and typically at least ten bases that are complementary to, and a 3′ terminal base exactly complementary, to a first region of a second allelic DNA sequence haplotype element, and a second reverse primer whose 3′ end has at least seven and typically at least ten bases that are complementary to, and a 3′ terminal base exactly complementary to, a second region of said second allelic DNA sequence haplotype element, where said second forward primer and said second reverse primer are capable of participating together in a polymerase chain reaction; and in conjunction with the performance of said at least two polymerase chain reactions, monitoring of the amount of amplicon produced in each reaction by measuring fluorescent emission from a quantitation reagent sensitive to the amount of amplicon produced in the reaction, where such monitoring occurs automatically no less often than every fifth polymerase chain reaction temperature cycle, and comparing the amount of fluorescence in each reaction to determine whether the corresponding haplotype is present in the input sample containing DNA.

### BRIEF DESCRIPTION OF THE DRAWINGS

FIG. 1 shows the Cₜs (average of two replicates) from protocol 1 in Example 1.

FIG. 2 shows the same data expressed as ΔCt.

FIG. 3 shows the results of a DAP experiment with Delta-Taq polymerase.

### DETAILED DESCRIPTION OF THE INVENTION

Definitions

“Complementary” in reference to a primer and template refer to the existence of sufficient base sequence complementarity so that the primer can anneal to the template under particular conditions. For instance, in a PCR, primers are complementary to templates if they anneal under the conditions of the reaction.

“Exactly complementary” in reference to a primer and template refers to the matching of adenosine and adenosine analogs only with thymine and thymine analogs, and the matching of guanosine and guanosine analogs only with cytosine and cytosine analogs.

“Efficiency” in the context of a nucleic acid modifying enzyme of this invention refers to the ability of the enzyme to perform its catalytic function under specific reaction conditions. Typically, “efficiency” as defined herein is indicated by the amount of modified bases generated by the modifying enzyme per binding to a nucleic acid.

“Enhances” in the context of an enzyme refers to improving the activity of the enzyme, i.e., increasing the amount of product per unit enzyme per unit time.

“Fused” refers to linkage by covalent bonding.

“Heterologous”, when used with reference to portions of a protein, indicates that the protein comprises two or more domains that are not found in the same relationship to each other in nature. Such a protein, e.g., a fusion protein, contains two or more domains from unrelated proteins arranged to make a new functional protein.

“Polymerase” refers to an enzyme that performs template-directed synthesis of polynucleotides. A “polymerase” can include an entire enzyme or a catalytic domain.

“Domain” refers to a unit of a protein or protein complex, comprising a polypeptide subsequence, a complete polypeptide sequence, or a plurality of polypeptide sequences where that unit has a defined function. The function is understood to be broadly defined and can be ligand binding, catalytic activity or can have a stabilizing effect on the structure of the protein.

“Processivity” refers to the ability of a nucleic acid modifying enzyme to remain attached to the template or substrate and perform multiple modification reactions. Typically “processivity” refers to the number of reactions catalyzed per binding event.

“Self-quenched primers” or “self-quenching primers” refers to synthetic oligonucleotides containing an attached fluorophore, where such oligonucleotedes are designed and synthesized such that when the oligonucleotide is not paired with a complement, the fluorophore has lower fluorescence relative to the fluorophore of the same oligonucleotide when it is paired with its complement.

“Sequence-non-specific nucleic-acid-binding domain” refers to a protein domain which binds with significant affinity to a nucleic acid, for which there is no known nucleic acid which binds to the protein domain with more than 100-fold more affinity than another nucleic acid with the same nucleotide composition but a different nucleotide sequence.

“Thermally stable polymerase” as used herein refers to any enzyme that catalyzes polynucleotide synthesis by addition of nucleotide units to a nucleotide chain using DNA or RNA as a template and has an optimal activity at a temperature above 45° C.

“Thermus polymerase” refers to a family A DNA polymerase isolated from any Thermus species, including without limitation Thermus aquaticus, Thermus brockianus, and Thermus thermophilus; any recombinant enzymes deriving from Thermus species, and any functional derivatives thereof, whether derived by genetic modification or chemical modification or other methods known in the art.

The term “amplification reaction” refers to any in vitro means for multiplying the copies of a target sequence of nucleic acid. Such methods include but are not limited to polymerase (PCR), DNA ligase, (LCR), QβRNA replicase, and RNA transcription-based (TAS and 3SR) amplification reactions (See, e.g. Current Protocols in Human Genetics Dracopoli et al. eds., 2000, John Wiley & Sons, Inc.).

“Amplifying” refers to a step of submitting a solution to conditions sufficient to allow for amplification of a polynucleotide if all of the components of the reaction are intact. Components of an amplification reaction include, e.g., primers, a polynucleotide template, polymerase, nucleotides, and the like. The term “amplifying” typically refers to an “exponential” increase in target nucleic acid. However, “amplifying” as used herein can also refer to linear increases in the numbers of a select target sequence of nucleic acid.

The term “amplification reaction mixture” refers to an aqueous solution comprising the various reagents used to amplify a target nucleic acid. These include enzymes, aqueous buffers, salts, amplification primers, target nucleic acid, and nucleoside triphosphates. Depending upon the context, the mixture can be either a complete or incomplete amplification reaction mixture

“Polymerase chain reaction” or “PCR” refers to a method whereby a specific segment or subsequence of a target double-stranded DNA, is amplified in a geometric progression. PCR is well known to those of skill in the art; see, e.g., U.S. Pat. Nos. 4,683,195 and 4,683,202; PCR Technology: Principles and Applications for DNA Amplification (Erlich, ed., 1992) and PCR Protocols: A Guide to Methods and Applications, Innis et al., eds, 1990.

“Long PCR” refers to the amplification of a DNA fragment of 5 kb or longer in length. Long PCR is typically performed using specially-adapted polymerases or polymerase mixtures (see, e.g., U.S. Pat. Nos. 5,436,149 and 5,512,462, and WO01/92501) that are distinct from the polymerases conventionally used to amplify shorter products.

A “primer” refers to a polynucleotide sequence that hybridizes to a sequence on a target nucleic acid and serves as a point of initiation of nucleic acid synthesis. Primers can be of a variety of lengths and are often less than 50 nucleotides in length, for example 12-25 nucleotides, in length. The length and sequences of primers for use in PCR can be designed based on principles known to those of skill in the art, see, e.g., Innis et al., supra.

A “target” or “target sequence” refers to a single or double stranded polynucleotide sequence sought to be amplified in an amplification reaction. Two target sequences are different if they comprise non-identical polynucleotide sequences.

The term “subsequence” when referring to a nucleic acid refers to a sequence of nucleotides that are contiguous within a second sequence but does not include all of the nucleotides of the second sequence.

A “temperature profile” refers to the temperature and lengths of time of the denaturation, annealing and/or extension steps of a PCR reaction. A temperature profile for a PCR reaction typically consists of 10 to 60 repetitions of similar or identical shorter temperature profiles; each of these shorter profiles may typically define a two step or three-step PCR reaction. Selection of a “temperature profile” is based on various considerations known to those of skill in the art, see, e.g., Innis et al., supra. In a long PCR reaction as described herein, the extension time required to obtain an amplification product of 5 kb or greater in length is reduced compared to conventional polymerase mixtures.

PCR “sensitivity” refers to the ability to amplify a target nucleic acid that is present in low copy number. “Low copy number” refers to 10⁵, often 10⁴, 10³, 10², or fewer, copies of the target sequence in the nucleic acid sample to be amplified.

A “template” refers to a double stranded polynucleotide sequence that comprises the polynucleotide to be amplified, flanked by primer hybridization sites. Thus, a “target template” comprises the target polynucleotide sequence flanked by hybridization sites for a 5′ primer and a 3′ primer.

The term “query” position refers to the target polymorphic nucleotide or other polymorphism targeted by an assay of the invention.

The term “fluorophore” refers to chemical compounds which, when excited by exposure to particular wavelengths of light, emit light (i.e., fluoresce) at a different wavelength.

A “polymorphism” is an allelic variant. Polymorphisms can include single nucleotide polymorphisms as well as simple sequence length polymorphisms. A polymorphism can be due to one or more nucleotide substitutions at one allele in comparison to another allele or can be due to an insertion or deletion.

A “haplotype” consists of at least two polymorphisms linked together on the same chromosome, where each of the polymorphisms can occur, at least conceptually, independently of the other. Each of the polymorphisms that constitute a haplotype may individually be referred to as a “haplotype element.”

Introduction

The current invention provides methods of performing DNA polymorphism assays, wherein a specific assay for a particular polymorphism can be set up for the cost of synthesizing as few as three or four primer oligonucleotides, run with little more difficulty and for little more marginal cost than two small-scale PCR reactions, using only common laboratory equipment. The assay combines allele-specific PCR with some of the technology used for quantitative PCR, along with the surprising observation that simple rules are sufficient to design reliable assays with little or no experimentation. The method of the invention may be referred to as DAP, for Differential Amplification of Polymorphisms. In one aspect of the invention, DAP is used to score the presence of absence of particular polymorphisms in a DNA sample. In a further aspect of the invention, DAP is used to score the presence of absence of particular haplotypes in a DNA sample.

Scoring DNA polymorphisms

A first aspect of the invention is a method of scoring of polymorphisms in a DNA sample that is based on the surprising discovery that ASP using only a single difference between the two primers, corresponding to a polymorphic site, in combination with QPCR using a fluorescent readout, is sufficient to score all tested polymorphisms. Previous experience with ASP had suggested that a single difference between primers did not provide sufficient specificity for scoring polymorphisms and little has been done combining QPCR and ASP for polymorphism scoring. Moreover, the prior art this has not used single differences between primers as a the basis of the ASP.

Scoring Haplotypes

DAP can be used in many cases to score haplotypes. The technique depends on at least two polymorphisms that differ between two haplotypes being close enough together, for instance less than 5000 bases, that PCR can be performed using a primer located at each site. The polymorphisms may be SNPs, but can also be insertions, deletions, rearrangements, or mutations affecting more than one base.

For each known haplotype being assayed (each target haplotype), a pair of primers is synthesized such that both primers have a perfect match to the target haplotype, but at least one of the two primers contains at least one mismatch to every other known haplotype. A separate PCR is performed for each target haplotype. Any given diploid individual is expected to score positively for no more than two of the target haplotypes.

As in other forms of DAP, positive and negative scores for target haplotypes are determined on the basis of ΔCₜ. For each DNA sample, the PCR producing the earliest Cₜ results in a positive diagnosis for the corresponding target haplotype. All target haplotypes not actually present in the sample result in significantly later Cₜ. The cycle threshold (Cₜ) value, represents the number of cycles required to generate a detectable amount of DNA. In the equipment used, a detectable amount of DNA, typically an amount that is 5 standard deviations, preferably 10 standard deviations above the background noise level, is approximately 1 ng.

In some cases, two polymorphisms defining a haplotype may be far apart, for instance, 5 to 10 kilobases. In this case, it is preferred that DAP be performed with a polymerase having high intrinsic processivity so that amplification can be achieved over the complete distance. Furthermore, it is possible that a spurious product may be produced for a target haplotype due to strand-switching during PCR. In certain types of heterozygotes, a partially-complete strand generated from a correct extension on one chromosome could, in a subsequent PCR cycle, anneal to a template derived from the other chromosome, thereby generating a product that mimics the target haplotype. However, this phenomenon does not materially affect the AC, for most amplicons. To minimize this effect, it is especially advantageous to use polymerases having a high intrinsic processivity, so that they are more likely to complete synthesis of amplicons in a single PCR cycle. In particular, polymerases modified to increase processivity such as those disclosed in co-pending application Ser. No. 09/870,353 and corresponding WO publication WO01/92501 are often used in both of the above applications.

Method for Detection of DNA Polymorphisms

One aspect of the invention is a method of distinguishing the presence or absence in an input sample containing DNA of at least two alternative target DNAs, e.g., two polymorphic allelic sequence elements, wherein those elements are at least seven bases and typically at least ten bases in length, and differ in sequence in at least one position. The method requires the performance of at least two polymerase chain reactions. The first reaction comprises a sample DNA comprising a target DNA sequence, a first forward primer that has at least seven bases and typically at least ten bases at the 3′ end that are exactly complementary to one of the alleles of the target DNA sequence element, and a reverse primer capable of participating with the first forward primer in a polymerase chain reaction. The second reaction comprises the DNA sample to be analyzed, a second forward primer that has at least seven bases and typically at least ten bases, at the 3′ end that are exactly complementary to another allele of a target DNA sequence element, and a reverse primer capable of participating with the second forward primer in a polymerase chain reaction.

The amount of amplicon produced in each reaction is typically monitored by measuring fluorescent emission from a quantitation reagent sensitive to the amount of double-stranded polynucleotide present in the reaction. The monitoring occurs automatically no less often than every fifth polymerase chain reaction temperature cycle. The amount of fluorescence in each reaction is then compared to detect whether the corresponding allele is present in the input sample containing DNA.

A second aspect of the invention is a method of distinguishing the presence or absence in an input sample containing DNA of at least two alternative target DNAs, e.g., two polymorphic allelic sequence elements, wherein those elements are at least seven bases and typically at least ten bases in length, and differ in sequence in at least one position. The method requires the performance of a polymerase chain reaction containing two self-quenching allele-specific primers. The reaction comprises a sample DNA comprising a target DNA sequence, a first forward self-quenching primer that has at least seven bases and typically at least ten bases at the 3′ end that are complementary to one of the alleles of the target DNA sequence element and whose 3′ end base is exactly complementary with a position at which the two alleles differ (a query position); a second forward self-quenching primer that has at least seven bases and typically at least ten bases at the 3′ end that are complementary to another allele of a target DNA sequence element and whose 3′ end base is exactly complementary with a position at which the two alleles differ, and a reverse primer capable of participating with the two forward primers in a polymerase chain reaction.

The amount of amplicon produced in each reaction is typically monitored by measuring fluorescent emission from the two self-quenching primers, which are synthesized using distinguishable fluorophores. The monitoring occurs automatically no less often than every fifth polymerase chain reaction temperature cycle. The amount of fluorescence from each self-quenching primer is then compared to detect whether the corresponding allele is present in the input sample containing DNA.

Method for Detection of Haplotypes

The invention also provides a method of distinguishing the presence or absence in a DNA sample of at least two allelic DNA sequence haplotypes, where the haplotypes comprise at least two allelic haplotype elements, each at least 10 bases in length and each of which differ in sequence in at least one position. The method requires the performance of at least two polymerase chain reactions. The first reaction comprises the sample DNA, a first forward primer that has a 3′ in which at least seven bases, typically at least ten bases, are complementary to a subsequence of a first element of a first haplotype allele and has a 3′ end base exactly complementary with a position at which the two alleles differ; and a first reverse primer that has a 3′ end with at least seven bases, typically at least ten bases, that are complementary to a subsequence of a second element of a first haplotype allele and has a 3′ end base exactly complementary with a position at which the two alleles differ, where the forward primer and the reverse primer are capable of participating together in a polymerase chain reaction. A second reaction comprises the DNA sample, a second forward primer that has a 3′ end with at least seven bases, typically at least ten bases, that are complementary to a subsequence of an element of a second haplotype allele and has a 3′ end base exactly complementary to a position at which the two alleles differ, and a second reverse primer that has a 3′ end that has at least seven bases, typically at least ten bases, that are complementary to a subsequence of a second element of the second haplotype allele and has a 3′ end base exactly complementary to a position at which the two alleles differ, where the forward primer and the reverse primer are capable of participating together in a polymerase chain reaction.

The amount of amplicon produced in each reaction is typically monitored by measuring fluorescent emission from a quantitation reagent sensitive to the amount of double-stranded polynucleotide present in the reaction. The amount of fluorescence in each reaction is then compared to detect whether the corresponding haplotype is present in the sample DNA.

Reagents

The primers for the amplification reactions are designed according to known algorithms. Typically, commercially available or custom software will use algorithms to design primers such that that annealing temperatures are close to melting temperature. Typically, the primers are at least 12 bases, more often 15, 18, or 20 bases in length. Primers are typically designed so that all primers participating in a particular reaction have melting temperatures that are within 5° C., and most preferably within 2° C. of each other. Primers are further designed to avoid priming on themselves or each other. Primer concentration should be sufficient to bind to the amount of target sequences that are amplified so as to provide an accurate assessment of the quantity of amplified sequence. Those of skill in the art will recognize that the amount of concentration of primer will vary according to the binding affinity of the primers as well as the quantity of sequence to be bound. Typical primer concentrations will range from 0.01 μM to 0.5 μM.

The primers can be directly labeled, e.g., with a fluorescent moiety. However, the reactions are often performed in the presence of a fluorescent signal generator, e.g., SYBR Green I, that binds double stranded DNA.

The polymerase reactions are incubated under conditions in which the primers hybridize to the target sequences and are extended by a polymerase. As appreciated by those of skill in the art, such reaction conditions may vary, depending on the target sequence and the composition of the primer. The amplification reaction cycle conditions are selected so that the primers hybridize specifically to the target sequence and are extended. Exemplary PCR conditions for particular primer sets are provided in the examples.

Fluorescence-based assays can also be used that rely for signal generation on fluorescence resonance energy transfer, or “FRET”, according to which a change in fluorescence is caused by a change in the distance separating a first fluorophore from an interacting resonance energy acceptor, either another fluorophore or a quencher. Combinations of a fluorophore and an interacting molecule or moiety, including quenching molecules or moieties, are known as “FRET pairs.” The mechanism of FRET-pair interaction requires that the absorption spectrum of one member of the pair overlaps the emission spectrum of the other member, the first fluorophore. If the interacting molecule or moiety is a quencher, its absorption spectrum must overlap the emission spectrum of the fluorophore. Stryer, L., Ann. Rev. Biochem. 47: 819-846 (1978); BIOPHYSICAL CHEMISTRY part II, Techniques for the Study of Biological Structure and Function, C. R Cantor and P. R. Schimmel, pages 448455 (W. H. Freeman and Co., San Francisco, U.S.A., 1980); and Selvin, P. R., Methods in Enzymology 246: 300-335 (1995). Efficient FRET interaction requires that the absorption and emission spectra of the pair have a large degree of overlap. The efficiency of FRET interaction is linearly proportional to that overlap. See Haugland, R. P. et al. Proc. Natl. Acad. Sci. USA 63: 24-30 (1969). Typically, a large magnitude of signal (i.e., a high degree of overlap) is required. FRET pairs, including fluorophore-quencher pairs, are therefore typically chosen on that basis.

A variety of labeled nucleic acid hybridization probes and detection assays that utilize FRET and FRET pairs are known. One such scheme is described by Cardullo et al. Proc. Natl. Acad. Sci. USA 85: 8790-8794 (1988) and in Heller et al. EP 0070685. It uses a probe comprising a pair of oligodeoxynucleotides complementary to contiguous regions of a target DNA strand. One probe molecule contains a fluorescent label, a fluorophore, on its 5′ end, and the other probe molecule contains a different fluorescent label, also a fluorophore, on its 3′ end. When the probe is hybridized to the target sequence, the two labels are brought very close to each other. When the sample is stimulated by light of an appropriate frequency, fluorescence resonance energy transfer from one label to the other occurs. FRET produces a measurable change in spectral response from the labels, signaling the presence of targets. One label could be a “quencher,” which in this application is meant an interactive moiety (or molecule) that releases the accepted energy as heat.

Polymerases

Polymerases are well-known to those skilled in the art. These include both DNA-dependent polymerases and RNA-dependent polymerases such as reverse transcriptase. At least five families of DNA-dependent DNA polymerases are known, although most fall into families A, B and C. There is little or no structural or sequence similarity among the various families. Most family A polymerases are single chain proteins that can contain multiple enzymatic functions including polymerase, 3′ to 5′ exonuclease activity and 5′ to 3′ exonuclease activity. Family B polymerases typically have a single catalytic domain with polymerase and 3′ to 5′ exonuclease activity, as well as accessory factors. Family C polymerases are typically multi-subunit proteins with polymerizing and 3′ to 5′ exonuclease activity. In E. coli, three types of DNA polymerases have been found, DNA polymerases I (family A), II (family B), and HI (family C). In eukaryotic cells, three different family B polymerases, DNA polymerases α, δ, and ε, are implicated in nuclear replication, and a family A polymerase, polymerase γ, is used for mitochondrial DNA replication. Other types of DNA polymerases include phage polymerases.

In some embodiments, for example, in embodiments in which long PCR is necessary, it may be advantageous to use polymerases having enhanced processivity. Examples of these include those described in WO01/92501. These improved polymerases exhibit enhanced processivity due to the presence of a sequence-non-specific double-stranded DNA binding domain that is joined to the polymerase or the enzymatic domain of the polymerase). Often the binding domain is from a thermostable organism and provides enhanced activity at higher temperatures, e.g., temperatures above 45° C. For example, Sso7d and Sac7d are small (about 7,000 kd MW), basic chromosomal proteins from the hyperthermophilic archaeabacteria Sulfolobus solfataricus and S. acidocaldarius, respectively (see, e.g., Choli et al., Biochimica et Biophysica Acta 950:193-203, 1988; Baumann et al., Structural Biol. 1:808-819, 1994; and Gao et al, Nature Struc. Biol. 5:782-786, 1998). These proteins bind DNA in a sequence-independent manner and when bound, increase the TM of DNA by up to 40° C. under some conditions (McAfee et al., Biochemistry 34:1.0063-10077, 1995). These proteins and their homologs are often used as the sequence-non-specific DNA binding domain in improved polymerase fusion proteins.

In specific embodiments, Taq polymerases may be used in the methods of the invention. In particular, polymerase variants such as ΔTaq, which is a genetically modified version of standard Taq DNA polymerase that lacks the 5′ to 3′-exonuclease activity (Lawyer et al., J Biol Chem 264:6427-6437 (1989)), are often used in the methods of the invention. Taq polymerase domains may be incorporated into improved polymerase fusion proteins, e.g., Sso7d-ΔTaq fusion proteins as described (see, e.g., WO01/92501).

Other family A polymerases, including, but not limited to those that act similarly to Taq, e.g., Thermus brockianus polymerase, which is about 90% similar to Taq polymerase, as well as Thermus flavus polymerase, and Thermus thermophilus polymerase may also be used in the invention. In some embodiments, these polymerase lack the 5′ to 3′ exonuclease domain. Additionally, less extremely thermophilic polymerases, such as the family A polymerase from Bacillus stearothermophilus are likely to prove useful. Non-thermostable polymerases, such as E. coli Pol I may also be used where the reaction is not performed at high temperatures.

In other embodiments, Family B polymerases such as Pyrococcus polymerases, e.g., Pfu polymerase, may also be used, often as a polymerase domain that is fused to a sequence non-specific double-stranded nucleic acid binding domain, e.g., Sso7d domain. Any polymerase can be tested in this assay system, in particular to identify those that have a reduced propensity to extend a mismatched 3′ nucleotide.

Comparing the Level of Fluorescence

The presence of a particular allele, e.g., SNP or haplotype, in a nucleic acid sample is determined by assessing the fluorescence in a polymerase reaction for the particular allele in comparison to the fluorescence in a polymerase reaction for a second allele.

Comparing the amount of fluorescence, as used herein, refers to any method of determining differences between reactions based on increases or decreases in fluorescence. The reactions being compared may be in separate volumes, for instance in separate vessels, in which case the fluorophores measured may be the same; or the reactions being compared may be in the same volume, in which case the fluorophores measured typically differ in at least one spectral characteristic, such as excitation wavelength, emission wavelength, fluorescence lifetime, polarization, etc.

In some cases, the total amount of fluorescence may be measured, and the comparison may be on the basis of intensity of fluorescence, e.g., the intensity at the end of the reaction. More often, the comparison is on the basis of how early in a PCR the fluorescence reaches a threshold level.

Initial copy number can be quantified during real-time PCR analysis based on threshold cycle. See, Higuchi, R., et al. Biotechnology 11:1026-1030 (1993). Threshold cycle, or Ct, is defined as the cycle at which fluorescence is determined to be statistically significant above background. The cycle threshold is inversely proportional to the log of the initial copy number. The more template that is present to begin with, the fewer the number of cycles it takes to get to a point where the fluorescent signal is detectable above background.

When comparing reactions in which the fluorescence is generated by the same chemistry in each reaction, for example, the same fluorophores are used, direct measurement of the fluorescence can typically measure the reaction. However, when different chemistries, for instance different fluorophores, are used, some adjustment may need to be made, either in the chemistries (e.g., by adjusting concentrations) or in the measurements (e.g., via software) to assure that similar signals from the two chemistries will represent similar progress in the reactions. Furthermore, allele-specific QPCR depends on a delay in reaching a threshold value when a mismatch occurs in a primer, but the exact number of cycles of delay differ depending on the sequence of the locus being assayed and enzymological differences among polymerases. Therefore, each specific assay may need to be validated using known homozygous and heterozygous DNA in order to establish how many cycles of delay (between the cycle where the signal corresponding to a first allele reaches a threshold and the cycle where the signal corresponding to a second allele reaches a threshold) are needed to be able to definitively score a sample as homozygous for the first allele. Exemplary methods of determining ΔCt values that are indicative of the presence of an allele in a homozygote or heterozygote are provided in Examples 2 and 3.

All publications, patents, and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

### EXAMPLES

The following examples are provided by way of illustration only and not by way of limitation. Those of skill will readily recognize a variety of non-critical parameters that could be changed or modified to yield essentially similar results.

### Example 1

In this example it is demonstrated that the method of the invention is capable of distinguishing all four nucleotides at a single position.

In a typical SNP assay, there are only two known possible bases at the SNP site, and therefore only two allele-specific primers would be used, corresponding to those two bases.

However, to demonstrate the generality of the method, four templates were generated that were identical except for a single position, where each of the templates had a different base. Sixteen polymerase chain reactions were then performed, using all combinations of the four templates and four allele-specific primers, each complementary to a different version of the sequence.

A 475 bp portion of the human cytochrome P450 gene CYP2D6 (GenBank Accession # M33388, nucleotides 3265-3729) was amplified using primers A1 (forward) and A5 (reverse), and cloned into a TA cloning vector (Invitrogen Corp., Carlsbad Calif.). Three PCR primers identical in sequence to primer A1 except for a single difference at their 3′ terminal bases, primers A2, A3, and A4, were used to re-amplify the CYP2D6 fragment, along with reverse primer A5, to generate the point mutations A, C, and T at nucleotide 3280 (G is present in the most common allele in most human populations). The three amplicons were cloned as above.

In order to understand the effect of amplicon size, an additional reverse primer, A6, was designed to generate a much smaller amplicon of 57 bp.

Forward primer sequences:

| | |
|---|---|
| Primer A1: | AGG CGC TTC TCC GTG |
| Primer A2: | AGG CGC TTC TCC GTC |
| Primer A3: | AGG CGC TTC TCC GTA |
| Primer A4: | AGG CGC TTC TCC GTT |

Reverse primer sequences:

| | |
|---|---|
| Primer A5: | ATG TCC TTT CCC AAA CCC AT |
| Primer A6: | CTC CAG CGA CTT CTT GC |

DAP was performed using a DNA Engine Opticon Continuous Fluorescence Detection Thermal Cycler (MJ Research, Inc., Waltham Mass.). All reactions contained the double-stranded DNA-dependent fluorescent dye SYBR Green I. Increase in fluorescence was used to trace the increase in DNA amount in each cycle. Opticon Monitor software (MJ Research, Inc., Waltham Mass.) was used to determine the threshold cycle for each reaction.

Reactions were performed in 32 sets of duplicates, with each set containing 300 nM of one of primers A1-A4 and 300 nM of one of primers A5 and A6. Each reaction also contained 10⁵ copies of one of the four the template DNA clones.

Protocol 1:

10 μl reactions were set up, each containing:
5 μl 2× reagent mixture (QuantiTect SYBR Green PCR Kit (Qiagen, Inc., Valencia Calif.))
5 μl template and primer mixture as described above.
   Reactions were thermally cycled as follows:
Step 1: 95° C. for 15 min
Step 2: 95° C. for 5 sec
Step 3: 60° C. for 15 sec
Step 4: 72° C. for 45 sec and read fluorescence
Step 5: Go to step 2 39 times.
   A melting curve analysis was performed following each PCR:
72° C.-95° C., 0.2 C increment, hold for 10 sec followed by a signal read

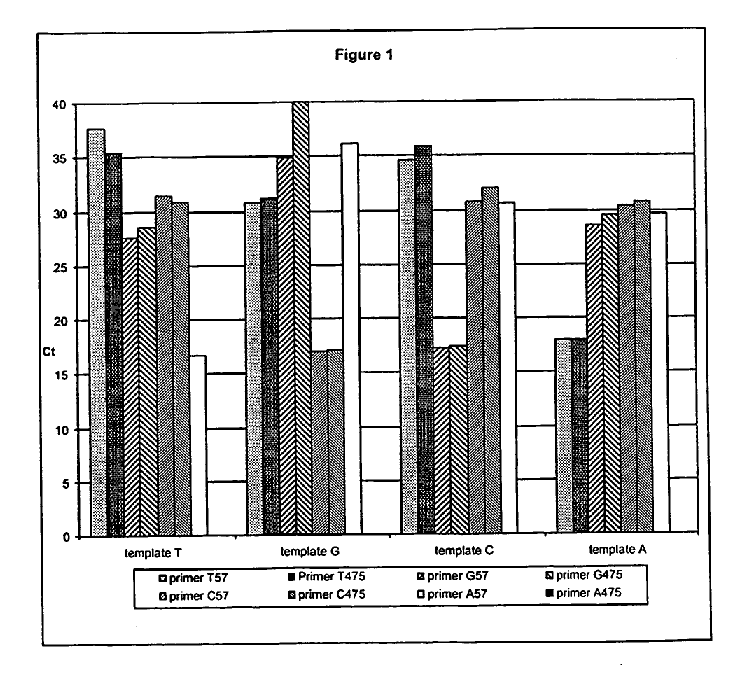

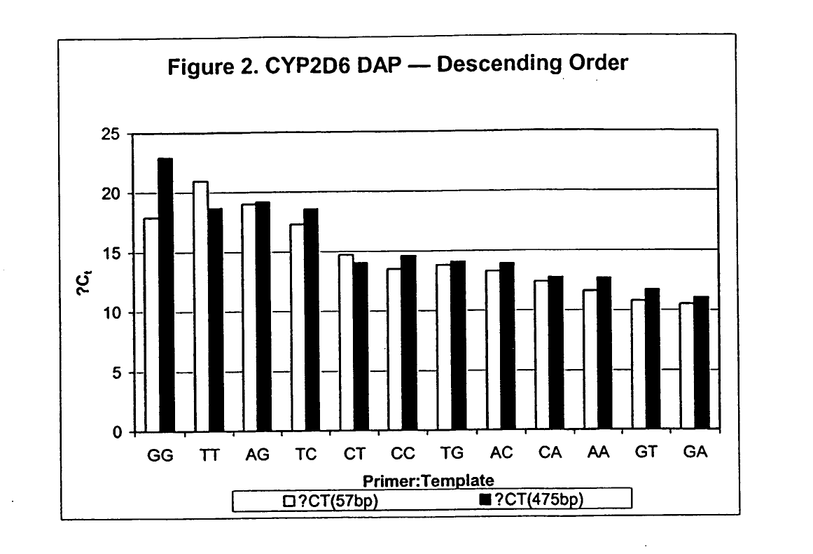

FIG. 1 shows the Cₜs (average of two replicates) from protocol 1. All perfect match conditions gave much lower Cₜ than all mismatch conditions. Long (475 bp) and short (57 bp) amplicons gave very similar results. FIG. 2 shows the same data expressed as ΔCₜ. For each template and amplicon length, the Cₜ value for the perfect match condition was subtracted from all Cₜ values, and Cₜ values were arranged in descending order. Minimum ΔC₁ values were greater than 10.

Protocol 2:

It has been reported that derivatives of Taq polymerase with the 5′-3′ exonuclease domain removed (generally termed “delta-Taq” and including “Stoffel fragment” polymerase available from ABI, Foster City, Calif.) are less likely to extend a 3′ mismatch.

The experiment of Protocol 1 was repeated, except that the reaction mixtures contained:
10 mM Tris-Cl pH 8.8
10 mM KCl
2.5 mM MgCl₂
1× SYBR Green I dye
0.1 U/μl StH enzyme (Stoffel fragment polymerase with histidines attached for purification)
200 μM each dNTP
Final volume was 20 μl.
   Reactions were thermally cycled as follows:
Step 1: 95° C. for 15 min
Step 2: 95° C. for 5 sec
Step 3: 60° C. for 15 sec
Step 4: 72° C. for 45 sec and read fluorescence
Step 5: Go to step 2 39 times.

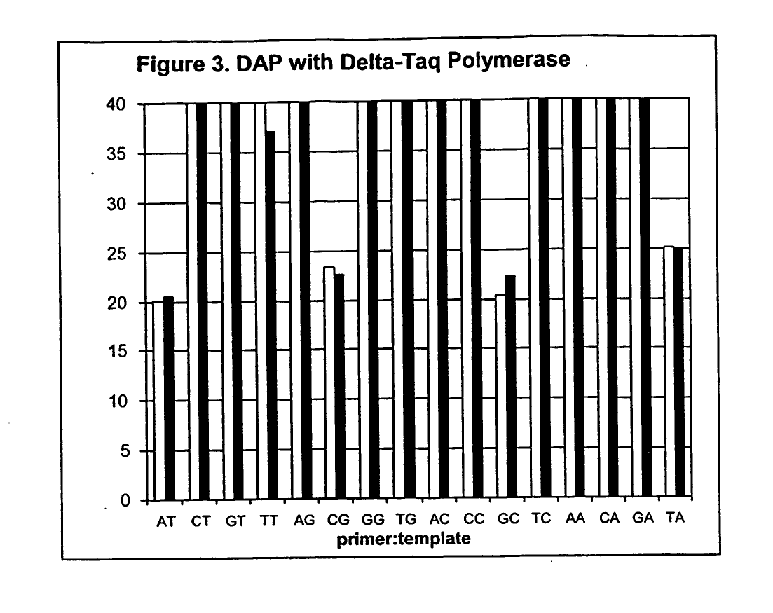

Results are shown in FIG. 3. Data from both replicates are show, and are reproducible. The minimum ΔCₜ for any mismatch, condition relative to the perfect match on the same template was 14.9. Many of the ΔCₜ values would have been higher, but the experiment was terminated at 40 cycles. The experiments shows that StH was effective at distinguishing correctly base-paired primers from incorrectly base-paired primers.

### Example 2

This example illustrates the use of DAP to distinguish haplotypes. The haplotypes distinguished are three alleles of human Apolipotein E (ApoE) which have been shown to affect risk for atherosclerosis and Alzheimer's disease.

The ApoE gene (GenBank Acc# XM_{—}044325) has been functionally characterized into three alleles; however, each allele is defined by the bases present at two separate positions, and as such the alleles are actually haplotypes. The SNPs making up the haplotypes are present at two sites within the ApoE gene: T/C at base 446 and C/T at base 584, corresponding to the protein sequence changes of Cys/Arg at amino acid 112 and Arg/Cys at amino acid 158. T446 with C584 is the E3 allele, T466 with T584 is the E2 allele, and C466 with C584 is the E4 allele. No instances of C466 with T584 have been reported (reviewed by de Knijff et al., Human Mutation 4: 178-194, 1994). The combinations of these haplotypes yield six different genotypes: E2/E2, E3/E3. E4/E4, E2/E3, E2/E4, and E3/E4. To test the three haplotypes, four PCR primers were designed:

| | |
|---|---|
| Primer B1: | GGA CAT GGA GGA CGT GT |
| Primer B2: | GGA CAT GGA GGA CGT GC |
| Primer B3: | G GTA CAC TGC CAG GCG |
| Primer B4: | G GTA CAC TGC CA |

Primers B1 and B2 are forward primers and primers B3 and B4 are reverse primers. The size of the amplicon is 169 bp. Three combinations of the primers were used for the three ApoE haplotypes—primer B1 and B3 for the E3 haplotype, primer B1 and B4 for the E2 haplotype, and primer B2 and B3 for the E4 haplotype. The combination of primers B2 and B4 was also used for a negative control in some samples, since it was not expected to be found.

The DAP assay was validated by testing human genomic samples for ApoE haplotype. The DNA source was 110 anonymous human blood samples. All the individuals were randomly selected and of unknown genotype. DNA was isolated by a standard protocol.

DAP was performed using a DNA Engine Opticon Continuous Fluorescence Detection Thermal Cycler (MJ Research, Inc., Waltham Mass.). All reactions contained the double-stranded DNA-dependent fluorescent dye SYBR Green I. Increase in fluorescence was used to trace the increase in DNA amount in each cycle. Opticon Monitor software (MJ Research, Inc., Waltham Mass.) was used to determine the threshold cycle for each reaction.

Each of the 110 human DNA samples was tested with the combinations forward and reverse primers corresponding to E2, E3, and E4. Final primer concentrations were 300 nM each. Each reaction also contained 20 ng genomic DNA, about 12,000 haploid genomes. Reactions were performed in duplicate.
10 μl reactions were set up, each containing:
5 μl 2× reagent mixture (QuantiTect SYBR Green PCR Kit (Qiagen, Inc., Valencia Calif.))
5 μl mix of primers and genomic DNA, as described above.
   The thermal cycler program was:
Step 1: 95 C for 1 min
Step 2: 95 C for 5 sec
Step 3: 59 C for 15 sec
Step 4: 72 C for 30 sec
Step 5: Go to Step 2 35 times.

Melting curve analysis was performed following each PCR to confirm the quality of the PCR product. The procedure for the melting curve analysis was described in Example 1. A call for a haplotype is made only when at least one Cₜ is significantly earlier than one other Cₜ. If this is true, then haplotypes are called as follows:
1. If one Cₜ, is significantly earlier than the other two, the sample is called homozygous for the haplotype corresponding to the reaction with the early Cₜ.
2. If two Cₜs are significantly earlier than the other one, the sample is called heterozygous for the haplotypes corresponding to the reactions with the early Cₜ.
3. If no Cₜs are in the expected range for early Cₜs, the assay is scored as failed PCR, poor DNA sample quality, or none of the target haplotypes present in the sample (possible but unlikely in the case of ApoE).
   Results:

All assays produced an unambiguous haplotype determination. Genotype and allele frequency as determined in this example were compared with those reported by Peter de Knijff: (Human Mutation 4: 178-194, 1994), and results are shown in Table 1.

TABLE 1

| Genotypes | No. | Genotype % | Expected % | Alleles | No. | Frequency | de Knijff |
|---|---|---|---|---|---|---|---|
| E3/E3 | 73 | 66.36% | 62.55% | E2 | 14 | 6.36% | 0-14.5% |
| E4/E4 | 5 | 4.55% | 2.12% | E3 | 174 | 79.09% | 41-91.1% |
| E2/E2 | 2 | 1.82% | 0.40% | E4 | 32 | 14.55% | 6.4-36.8% |
| E3/E4 | 20 | 18.18% | 23.01% | | | | |
| E2/E3 | 8 | 7.27% | 10.07% | | | | |
| E2/E4 | 2 | 1.82% | 1.85% | | | | |
| Total | 110 | 100.00% | 100.00% | | 220 | 100.00% | |

As can be seen in Table 1, the observed allele frequency is very consistent with the reported ranges. In this sample, there was a slight excess of homozygotes over the number expected from Hardy-Weinberg equilibrium, indicating population sub-structure.

The results also suggest that strand-switching during PCR amplification is not a problem in this procedure. If strand-switching produced recombinant alleles with high frequency, then E2/E4 heterozygotes would also be positive for E3. This was not the case.

### Example 3

In this example, a single-tube assay for a SNP or a polymorphism is provided, using two differently-colored fluorophores and self-quenching primers.

The SNP scored was in the gene ApoE (see Example 2). Primer sequences were:

| | |
|---|---|
| C1 | 5′-(BHQ-1)-GGACATGGAGGACGTG(FAM-dT)-3′ |
| C2 | 5′-(BHQ-1)-GGACATGGAGGACGTG(HEX-dC)-3′ |
| C3 | 5′-GGACATGGAGGACGTG-3′ |

BHQ-1 is a commercially-available FRET quencher, Black Hole Quencher, FAM is Fluorescein, and HEX is hexachlorofluorescein. FAM and HEX are covalently attached directly to the bases, as is well known in the art. All modified oligonucleotides were purchased from Trilink Biotechnologies, Inc., San Diego Calif. C1 and C2 are self-quenched primers in the forward direction with a quencher on the 5′ end and a fluor on the 3′ base. When these are extended and incorporated into double-stranded DNA, quenching decreases. The 3′ nucleotides of C1 and C2 are query positions, and are complementary to common alleles of a SNP in ApoE. C3 is an unmodified primer in the forward direction that does not contain the query position.

Templates for PCR were 1 ng of the plasmids p770ApoE112T or p770ApoE112C, which contain T and C respectively at the query position, simulating DNA from homozygous individuals, or an equal mixtures thereof, to simulate a heterozygote.

Reactions contained 50 U/ml AmpliTaq polymerase and its recommended buffer containing 2.5 mM Mg⁺⁺ (ABI, Foster City Calif.), supplemented with 1M Betaine. Primers were used at 2.5 μM each.

Reactions were scored using a DNA Engine Opticon 2 (MJ Research) which can read the amount of fluorescence in two wavelength bands at arbitrary times during a PCR. Opticon Monitor Software (MJ Research) was used to perform color separation on the raw data to determine the amount of fluorescence corresponding to each fluorophore present, and to determine the Ct for each color using standard algorithms.

Cycling parameters were:

Step Action
1. Incubate at 96° C. until manually advanced to next step.
2. Incubate at 96° C. for 00:01:00
3. Incubate at 96° C. for 00:00:15
4. Read fluorescence
5. Incubate at 60° C. for 00:00:30
6. Read fluorescence
7. Goto step 2 for 40 more times
8. Incubate at 72° C. for 00:10:00
9. Melting Curve from 65° C. to 98.0 read every 0.2 hold 00:00:01
10. Incubate at 72° C. for 00:10:00
11. Incubate at 10° C. for 00:02:00

END

Results

| | Plasmid Templates | | |
|---|---|---|---|
| | p770ApoE112T | p770ApoE112C | Equal Mixture |
| Ct, FAM | 15.51 | >40 | 17.73 |
| channel (T) | | | |
| Ct, HEX | >40 | 18.01 | 19.97 |
| channel (C) | | | |

In this example, no attempt was made to equalize the signals for the two color channels either through chemistry adjustments or through software. However, the threshold signal levels for the two channels were determined independently. Nevertheless, the difference in Ct between the two channels for the equal mixture of templates was only slightly greater than 2, while the difference ins Ct between the two channels for the pure templates was greater than 20 in each case. Thus, the difference in Ct clearly distinguishes the two simulated homozygotes from each other and from the heterozygote.

What is claimed is:

1. A method of determining the presence or absence of a first allele of a DNA sequence element in a DNA sample, wherein the element is at least seven bases in length and differs in sequence in at least one position from a second allele, the method comprising:
providing a first polymerase chain reaction chain reaction comprising the DNA sample, a first forward primer comprising a 3′ end having at least seven bases that are exactly complementary to the first allele, and a reverse primer that participates with the first forward primer in a polymerase chain reaction;
providing a second polymerase chain reaction comprising the DNA sample, a second forward primer comprising a 3′ end having at least seven bases that are exactly complementary to the second allele, and a reverse primer that participates with the second forward primer in a polymerase chain reaction;
incubating the first and second polymerase chain reactions under cycle conditions in which the primers are extended by a polymerase;
monitoring of the amount of amplicon produced in each reaction by measuring fluorescent emission from a quantitation reagent sensitive to the amount of double-stranded polynucleotide present in each reaction, wherein such monitoring occurs no less often than every fifth polymerase chain reaction temperature cycle, and
comparing the fluorescence in each reaction, thereby determining the presence or absence of a first allele in the sample.

2. The method of claim 1, wherein the polymerase is ΔTaq.

3. The method of claim 1, wherein the step of comparing the fluorescence in each reaction comprises determining the total amount of fluorescence in each reaction.

4. The method of claim 1, wherein the step of comparing the fluorescence in each reaction comprises determining the Cₜ.

5. A method of distinguishing the presence or absence in a DNA sample of at least two allelic DNA sequence haplotypes, wherein the two haplotypes each comprise at least two allelic haplotype elements, each haplotype element being at least seven bases in length and each allelic haplotype element differing in sequence from the corresponding element of the other haplotype allele in at least one position, the method comprising:
providing a first polymerase chain reaction comprising the input DNA sample, a first forward primer comprising a 3′ end sequence having at least seven bases that hybridizes to the first element of the first haplotype and a 3′ base exactly complementary to a first element of a first haplotype, and a first reverse primer comprising a 3′end sequence having at least seven bases that hybridizes to the second element of the first haplotype and a 3′ base exactly complementary to the second element of the first haplotype, wherein said first forward primer and said first reverse primer participate together in a polymerase chain reaction; and
providing a second polymerase chain reaction comprising the input sample containing DNA, a second forward primer comprising a 3′ end sequence having at least seven bases that hybridizes with the first element of the second haplotype and a 3′ base exactly complementary to the first element of the second haplotype, and a second reverse primer comprising a 3′ end sequence having at least seven bases that hybridizes with the second element of the second haplotype and a 3′ base exactly complementary to the second element of the second haplotype, wherein said second forward primer and said second reverse primer participates together in a polymerase chain reaction; and
incubating the first and second polymerase chain reactions under temperature cycling conditions in which the primers are extended by a polymerase;
monitoring of the amount of amplicon produced in each reaction by measuring fluorescent emission from a quantitation reagent sensitive to the amount of double-stranded polynucleotide present in the reaction, wherein such monitoring occurs automatically no less often than every fifth polymerase chain reaction temperature cycle, and
comparing the fluorescence in each reaction, thereby determining whether the first haplotype or the second haplotype is present in the input sample containing DNA.

6. The method of claim 5, wherein the polymerase is ΔTaq.

7. The method of claim 5, wherein the allelic haplotype elements are at least 5000 bp apart.

8. The method of claim 7, further wherein the polymerase is Sso7d-ΔTaq.

9. The method of claim 5, where the step of comparing the fluorescence in each reaction comprises determining the total amount of fluorescence.

10. The method of claim 5, wherein the step of comparing the fluorescence in each reaction comprises determining the Cₜ.

11. A method of distinguishing the presence or absence in a DNA sample of at least two alternative alleles of a DNA sequence element, wherein the element is at least seven bases in length and the two alleles differ in sequence in at least one position, the method comprising:
providing a polymerase chain reaction comprising the input sample containing DNA,
a first forward self-quenched primer comprising a first fluorophore, said primer having its 3′ at least seven bases that hybridize to the first allele of the DNA sequence element and its 3′ terminal base exactly complementary to said first allele of the DNA sequence element,
a second forward self-quenched primer comprising a second fluorophore, said primer having its 3′ at least seven bases that hybridize to the second allele of the DNA sequence element and its 3′ terminal base exactly complementary to said second allele of the DNA sequence element, said first and second fluorophores being distinguishable by their spectral characteristics, and
a reverse primer that participates with the first and second forward primers in a polymerase chain reaction;
incubating the reaction under temperature cycling conditions in which the primers are extended by a polymerase; and
monitoring of the amount of amplicon produced using each self-quenched primer by measuring fluorescent emission from each of said fluorophores, where such monitoring occurs automatically no less often than every fifth polymerase chain reaction temperature cycle, and comparing the amount of fluorescence corresponding to each self-quenched primer to determine the presence or absence of the allele in the input sample containing DNA.

12. The method of claim 11, wherein the polymerase is ΔTaq.

13. The method of claim 11, where the step of comparing the fluorescence in each reaction comprises determining the total amount of fluorescence.

14. The method of claim 11, wherein the step of comparing the fluorescence in each reaction comprises determining the Cₜ.


SEQUENCE LISTING (13 sequences)

SEQ ID NO: 1 - DNA, 15 bp, Artificial Sequence
  Description of Artificial Sequencedifferential amplification of polymorphisms (DAP) forward primer A1
aggcgcttctccgtg

SEQ ID NO: 2 - DNA, 15 bp, Artificial Sequence
  Description of Artificial Sequencedifferential amplification of polymorphisms (DAP) forward primer A2
aggcgcttctccgtc

SEQ ID NO: 3 - DNA, 15 bp, Artificial Sequence
  Description of Artificial Sequencedifferential amplification of polymorphisms (DAP) forward primer A3
aggcgcttctccgta

SEQ ID NO: 4 - DNA, 15 bp, Artificial Sequence
  Description of Artificial Sequencedifferential amplification of polymorphisms (DAP) forward primer A4
aggcgcttctccgtt

SEQ ID NO: 5 - DNA, 20 bp, Artificial Sequence
  Description of Artificial Sequencedifferential amplification of polymorphisms (DAP) reverse primer A5
atgtcctttcccaaacccat

SEQ ID NO: 6 - DNA, 17 bp, Artificial Sequence
  Description of Artificial Sequencedifferential amplification of polymorphisms (DAP) reverse primer A6
ctccagcgacttcttgc

SEQ ID NO: 7 - DNA, 17 bp, Artificial Sequence
  Description of Artificial SequencePCR forward primer B1
ggacatggaggacgtgt

SEQ ID NO: 8 - DNA, 17 bp, Artificial Sequence
  Description of Artificial SequencePCR forward primer B2
ggacatggaggacgtgc

SEQ ID NO: 9 - DNA, 16 bp, Artificial Sequence
  Description of Artificial SequencePCR reverse primer B3
ggtacactgccaggcg

SEQ ID NO: 10 - DNA, 12 bp, Artificial Sequence
  Description of Artificial SequencePCR reverse primer B4
ggtacactgcca

SEQ ID NO: 11 - DNA, 17 bp, Artificial Sequence
  Description of Artificial Sequence self-quenched forward primer C1
ngacatggaggacgtgn

SEQ ID NO: 12 - DNA, 17 bp, Artificial Sequence
  Description of Artificial Sequence self-quenched forward primer C2
ngacatggaggacgtgn

SEQ ID NO: 13 - DNA, 16 bp, Artificial Sequence
  Description of Artificial Sequenceunmodified forward primer C3
ggacatggaggacgtg